(19) United States
(12) Patent Application Publication
(10) Pub. No.: US 2019/0365402 A1
(43) Pub. Date: Dec. 5, 2019

(54) MINIMALLY INVASIVE DEVICE AND SYSTEM

(71) Applicant: Human Xtensions Ltd., Netanya (IL)

(72) Inventors: Mordehai SHOLEV, Moshav Amikam (IL); Gilad HEFTMAN, Pardes Hana Karkur (IL); Assaf KAUFMAN, Tal Shahar (IL)

(73) Assignee: Human Xtensions Ltd., Netanya (IL)

(21) Appl. No.: 16/479,595

(22) PCT Filed: Jan. 23, 2018
(86) PCT No.: PCT/IL2018/050083

§ 371 Date: Jul. 21, 2019

Related U.S. Application Data
(60) Provisional application No. 62/449,186, filed on Jan. 23, 2017.

(51) Int. Cl. A61B 17/29; A61B 1/00; A61B 17/34

(52) U.S. Cl. CPC A61B 17/29; A61B 1/00133; A61B 17/3421; A61B 2017/00398; A61B 2017/3454; A61B 2017/3445; A61B 1/0016

(57) ABSTRACT

An introducer device and system are provided. The introducer includes an elongated body including at least one lumen sized and configured for supporting delivery of a medical device therethrough. The first portion of the elongated body is steerable and a second portion of the elongated body is telescopically extendable and retractable.

### FIELD AND BACKGROUND OF THE INVENTION

The present invention relates to a device and system for use in minimally invasive surgery and, more particularly, to a steerable introducer, or an introducer system that can be used along with a surgical instrument to perform minimally invasive procedures in a body cavity or lumen.

Medical devices such as endoscopes and catheters are widely used in minimally invasive procedures for viewing or treating organs, cavities, passageways, and tissues. Generally, such devices include an elongated device body which is designed for delivering and positioning a distally-mounted instrument (e.g. scalpel, grasper or camera/camera lens) within a body cavity, vessel or tissue.

Since such devices are delivered through a delivery port which is positioned through a small incision made in the tissue wall (e.g. abdominal wall), or through a natural orifice and are utilized in an anatomically constrained space, it is desirable that the medical device or at least a portion thereof be steerable, or maneuverable inside the body using controls positioned outside the body (at the proximal end of the medical device). Such steering enables an operator to guide the device within the body and accurately position the distally-mounted instrument at an anatomical landmark.

Although steerable devices considerably enhance the ability of a surgeon to accurately position a distally-mounted instrument at an anatomical landmark, they are large and heavy and require long and complicated setup procedures. In addition, most steerable devices have a limited range of movement and utilize large interface consoles which distance the surgeon from the patient and require support staff.

Thus, there is a need for a minimally invasive device and system which can be used to more accurately position an effector at an anatomical landmark within a body cavity while being free of the aforementioned limitations of prior art devices.

### SUMMARY OF THE INVENTION

According to one aspect of the present invention there is provided a system for minimally invasive procedures comprising: (a) a first device having an elongated body including a first lumen having a distal opening, at least a portion of the elongated body being steerable; (b) a second device being positionable within the first lumen with a distal portion thereof protruding from the distal opening; and (c) a support frame and a rail being independently couplable to proximal portions of the first device and the second device, wherein the rail is configured such that the second device is movable with respect to the first device along a longitudinal axis of the rail.

According to further features in preferred embodiments of the invention described below, the distal portion of the second device is steerable.

According to still further features in the described preferred embodiments the system further comprising a first motor pack attachable to a proximal end of the first device and a second motor attachable to a proximal end of the second device.

According to still further features in the described preferred embodiments the first motor pack is configured for steering the at least a portion of the elongated body.

According to still further features in the described preferred embodiments the second motor pack is configured for steering the distal portion of the second device.

According to still further features in the described preferred embodiments the elongated body is positionable within a body cavity/lumen of a subject through an access site.

According to still further features in the described preferred embodiments the second device includes a second lumen having a distal opening.

According to still further features in the described preferred embodiments the system further comprising a third device having a tool at a distal end thereof, the third device being positionable within the second lumen with the tool protruding from the distal opening of the second lumen.

According to still further features in the described preferred embodiments the tool is a grasper, needle or a snare.

According to still further features in the described preferred embodiments the rail includes a linear actuator for moving the second device with respect to the first device along the longitudinal axis.

According to still further features in the described preferred embodiments the second device includes a tool attached to the distal portion.

According to still further features in the described preferred embodiments the tool is a grasper, a needle holder or a hook.

According to still further features in the described preferred embodiments the at least the portion of the elongated body includes at least two independently steerable regions.

According to still further features in the described preferred embodiments the distal portion is 10-50 mm in length.

According to still further features in the described preferred embodiments the support frame is attachable to a bed or a floor stand.

According to still further features in the described preferred embodiments the first device includes at least one control knob for manually steering the at least the portion of the elongated body.

According to still further features in the described preferred embodiments the first device includes an irrigation lumen and a suction lumen.

According to another aspect of the present invention there is provided an introducer for minimally invasive surgery comprising an elongated body including at least one lumen sized and configured for supporting delivery of a medical device therethrough, wherein a first portion of the elongated body is steerable and a second portion of the elongated body is telescopically extendable and retractable.

According to still another aspect of the present invention there is provided a system including the introducer and the medical device positioned within the at least one lumen.

According to still further features in the described preferred embodiments the medical device includes a steerable distal portion.

According to still further features in the described preferred embodiments the distal portion is lockable to the second portion of the introducer, such that when the medical device is moved with respect to the introducer, the second portion of the elongated body is telescopically extended or retracted.

The present invention successfully addresses the shortcomings of the presently known configurations by providing a steerable introducer or introducer system which can be used in minimally invasive procedures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates an embodiment of the present system having two motorized introducers and a motorized surgical instrument attached to a rail of a support frame.

FIGS. 2A-C illustrate the external introducer (FIG. 2A), the internal introducer (FIG. 2B) and the surgical instrument (FIG. 2C) of the system of FIG. 1.

FIGS. 3A-D illustrate the external introducer (FIGS. 3A, 3D), its motor interface portion (FIG. 3B) and its shaft lumens (FIG. 3C).

FIG. 4 illustrates another embodiment of the present system having a motorized telescopic introducer attached to a support frame, and a motorized surgical instrument attached to a rail.

FIGS. 5A-B illustrate the rail (FIG. 5A) and its linear actuator portion (FIG. 5B).

FIG. 6 illustrates the various motorized (active) and non-motorized (passive) movements of the system of FIG. 4.

FIGS. 7A-B illustrate the telescopic introducer of the system of FIG. 4 and an access port tool (FIG. 7A) for positioning within a lumen of the telescopic introducer (FIG. 7B).

FIGS. 8A-C illustrate the telescopic introducer of the present invention showing the articulating region and motor pack connectors.

FIGS. 9A-D illustrate the surgical instrument motor pack (FIGS. 9A-B) and the introducer motor pack (FIGS. 9C-D), showing the front shafts (FIGS. 9A, 9C) and rear electrical connectors (FIGS. 9B, 9D).

FIGS. 10A-B illustrate a motor pack and electrical cable connector shown disconnected (FIG. 10A) and connected (FIG. 10B).

FIG. 11 illustrates the surgical instrument with motor pack and cable interface.

FIGS. 12A-B illustrate a surgical instrument positioned within a telescopic introducer (FIG. 12A) and a locking mechanism (FIG. 12B) for locking a shaft of the surgical instrument to a shaft of the telescopic introducer.

FIG. 13 illustrates an articulating region of the surgical instruments showing the articulation wires and a central cable that actuates the end effector.

FIGS. 14A-K illustrate the setup and use of the present system in a minimally invasive procedure.

FIG. 15 illustrates a prototype of an introducer-instrument system constructed in accordance with the teachings of the present invention.

### DESCRIPTION OF THE PREFERRED EMBODIMENTS

The present invention is of a device and system which can be used in a minimally invasive procedure. Specifically, the present invention can be used to introduce, steer and control surgical instruments in a minimally invasive procedure in a body cavity (e.g. abdominal cavity) or a lumen (e.g. GI tract).

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Surgical instrument having articulating regions steerable from outside the body are well known in the art. Such instruments are introduced into a body cavity or lumen through a natural (e.g. mouth or anal orifice) or user-generated access site (small incision in abdominal wall).

Compared to their non-steerable counterparts, steerable surgical instruments are more easily maneuvered to an anatomical site and as such, use of such instruments improves surgical accuracy and outcome while reducing procedure time.

While reducing the present invention to practice, the present inventor sought out to improve the maneuverability of steerable as well as non-steerable instruments by devising steerable introducers and introducer systems. As is further described hereinunder, the present introducers can be used to steer surgical instruments within a body cavity or lumen or enhance the maneuverability of steerable surgical instruments thus providing superior intrabody positioning of an effector end (grasper, needle, basket, balloon, camera, blade, snare and the like).

Thus according to one aspect of the present invention there is provided a device and system for minimally invasive procedure. As used herein, the phrase “minimally invasive procedure” refers to a surgical (therapeutic) or diagnostic procedure effected through a natural or created access site to a body cavity (e.g. abdominal, thoracic, cranial) or lumen (e.g. gastrointestinal tract, a duct or a vessel).

The present system includes one or more introducers (also referred to herein as first or second devices) and a surgical instrument (also referred to herein as third device).

The introducers have an elongated body (shaft) which includes one or more steerable portions (having articulating elements), while the surgical instrument can be a flexible non-steerable instrument (e.g. camera or optic fiber) or a steerable instrument having a shaft with one or more steerable portions.

Each introducer of the present invention includes a central lumen (also referred to herein as a first lumen) for accommodating an additional introducer (steerable or not) or a surgical instrument (steerable or not).

The introducer of the present invention can include additional lumens (second, third, fourth and fifth) for irrigation, aspiration, camera/optic fiber and additional surgical instruments.

The introducer can also include a telescopic portion that can be extended/retracted manually or via movement of an introducer or surgical instrument mounted therein. The latter can be achieved by locking a distal region of a surgical instrument (at a point proximal to steerable portion) to the telescopic portion of the introducer such that in and out movement of the surgical instrument within the introducer extends and collapses (respectively) the telescopic portion.

The steerable portion of the introducer(s) and surgical instrument of the present invention can be constructed from articulating links, a tube with cutout or the like. Numerous examples of steerable shaft portions are known in the art, see for example, U.S. Pat. Nos. 2,498,692; 4,753,223; 6,126,649; 5,873,842; 7,481,793; 6,817,974; 7,682,307 and U.S. Patent Application Publication No. 20090259141.

Deflection of the steerable portion is typically effected via one or more control wires which run along the shaft of the introducer to the distal end of the steerable portion.

The proximal end of each control wire is connected to a geared mechanism designed for pulling the wire to apply a force that deflects the steerable portion in the direction of the pulled wire. The geared mechanism can be actuated manually (via a knob or lever) or via an attached motor pack (with external electronic control).

The device effector end (distally-mounted instrument) is controlled via one or more additional wires which are similarly connected to the geared mechanism.

The introducer(s) and surgical instrument of the present system can be independently attached to a support frame which is in turn attached to a floor or fixture (e.g. bed) in a procedure room.

The support frame stabilizes the introducer(s) with respect to the access site and a rail mounted on the frame or introducer moves the surgical instrument in and out of the body cavity/lumen via a linear actuator.

The introducers and surgical instrument can be attached to the support frame and the rail in one of several configuration as follows:
(i) telescopic (steerable or non-steerable) introducer-non steerable surgical instrument; this configuration can be used in body cavities (e.g. laparoscopic surgery).
(ii) telescopic (steerable or non-steerable) introducer-non steerable camera; this configuration can be used in body cavities.
(iii) telescopic (steerable or non-steerable) introducer-steerable surgical instrument; this configuration can be used in body cavities.
(iv) steerable introducer-steerable surgical instrument; this configuration can be used in body cavities and natural orifices (e.g. endoscopic diagnostic procedures).
(v) steerable first introducer-steerable second introducer and steerable-non steerable surgical instrument; this configuration can be used in body cavities and natural orifices.

When combined into a system, the introducer(s) and surgical instrument each preferably include an attached motor pack for actuating steering and other functions (e.g. effector end of surgical instrument). Each motor pack is individually connected to (wired or via wireless communication), and controlled from, a user interface (e.g. the hand operated interface described in US20150164601, WO2015151093 or US20160184040). The user interface controls motor actuation to provide the following:
(i) deflection of the introducer steerable portion (right/left, up/down);
(ii) deflection of the instrument steerable portion (right/left, up/down);
(iii) in/out (zoom) movement of the instrument;
(iv) rotation of the instrument shaft or the rotation of the end effector tip (such as rotation of the end effector jaws or hook); and/or
(v) actuation of the end effector mechanism (such as open/close of end effector jaws.

Thus, the user interface provides three separate functions, positioning of the instrument shaft with respect to the tissue access site (by the introducer in/out, up/down, right/left, and steering), deflection of the distal steerable portion of the shaft, and actuation of a distally mounted effector end.

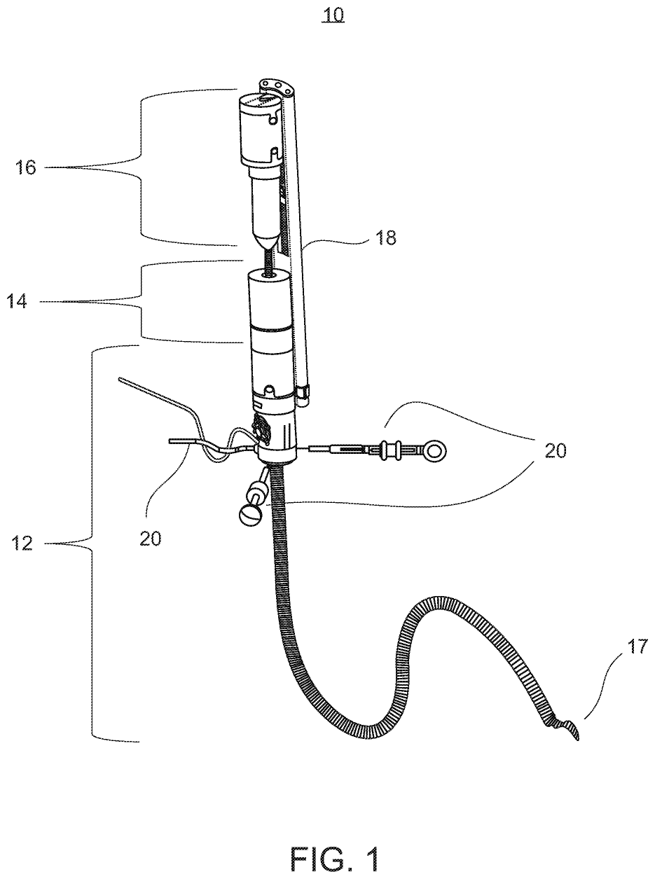

Referring now to the drawings, FIGS. 1-3D illustrate one embodiment of the present system which is referred to herein as system 10.

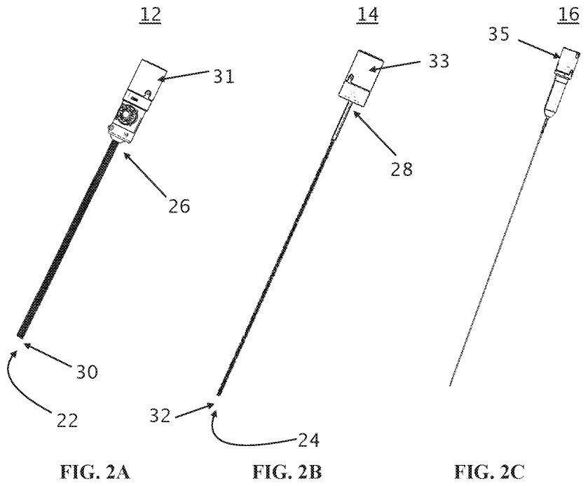

System 10 includes an external introducer 12 (shown separately in FIG. 2A), an internal introducer 14 (shown separately in FIG. 2B) and a surgical instrument 16 (shown separately in FIG. 2C) with an effector end 17 (cutting forceps).

External and internal introducers (12 and 14 respectively) each include one or more steerable portions while surgical instrument can be a flexible, non-steerable device such as endoscopic flexible biopsy forceps. In the configuration shown in FIGS. 1-3D, external introducer 12 includes a distal steerable portion with typical length of 150 mm, while internal introducer 14 includes a distal articulation portion which includes two or more independent steerable segments.

External introducer 12 and internal introducer 14 each include a lumen (22 and 24, FIGS. 2A and 2B respectively) having proximal (26—FIG. 2A, 28—FIG. 2B) and distal (30—FIG. 2A, 32—FIG. 2B) openings. Lumen 22 of external introducer is designed to accommodate internal introducer 14 while central lumen 24 of internal introducer 14 is designed to accommodate surgical instrument 16. The lumen opening include a seal (X-cut or O-ring) for sealing a shaft of a device (internal introducer 14 or surgical instrument 16) positioned within the lumen. Typical dimensions for external introducer 12 are length: 50-150 cm, diameter: 12-24 mm, distal steerable length: 30-150 mm and lumen diameter: 3-8 mm. External introducer 12 can be fabricated from a composite of rigid links metal coils and metal mesh with control cables/wires (for steering) disposed in or on the links.

Internal introducer 14 can be 70-155 cm in length, 3-6 mm in diameter with a distal steerable length of 20-40 mm, and a lumen diameter of 1.5-4 mm. Internal introducer 14 can be fabricated from a composite of rigid links metal coils and metal mesh. Control cables/wires (for steering) can be disposed in or on the links. Surgical instrument 16 can be an off-the-shelf instrument (e.g. biopsy forceps, electric biopsy forceps, grasping forceps, hook, snare, injection needle, hemoclip, balloon catheter) with a length of 100-180 cm and a diameter of 1.5-4 mm.

External introducer 12 can be attached directly to a support frame 20. Internal introducer 14 can be moved along the external introducer lumen or may be fixed to the most distal point, at the top of the external introducer motor pack 14. Surgical instrument 16 is mounted on rail 18.

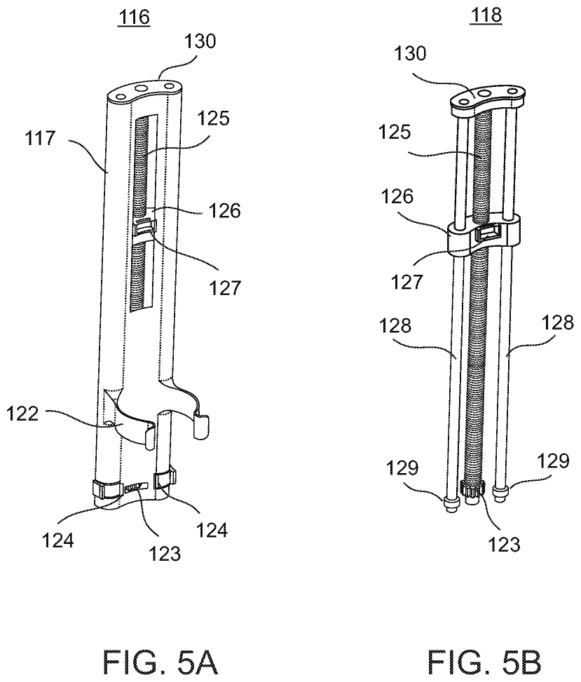

Rail 18 includes a linear actuator for translating devices attached thereto up and down (along a longitudinal axis thereof). Rail 18 and the linear actuator are described in detail hereinbelow with respect to FIGS. 5A-B.

In this configuration external introducer 12 is attached to frame 20, and internal introducer is fixed to any desired point along the external introducer lumen. Surgical instrument 16 is movable along rail 18 via the linear actuator moving in and out of internal introducer 14. Such a configuration enables a user to independently locate the external introducer in the operational site, adjust the position of internal introducer distal steerable portion relative to the external introducer distal end, adjust the height of surgical instrument 16 with respect to the access site and adjust the length of shaft protruding through the distal opening of the introducer (external or internal).

For example, by moving internal introducer 14 in and out of external introducer 12, a user can adjust the extent a shaft of internal introducer extends out of a distal opening of a central lumen of external introducer 12. By steering the distal portion of internal introducer 14 the surgeon positions the shaft of surgical instrument 16 at a desired angle with respect to the treated tissue. By moving surgical instrument 16 up/down along rail 18 enables positioning of an effector end with respect to tissue.

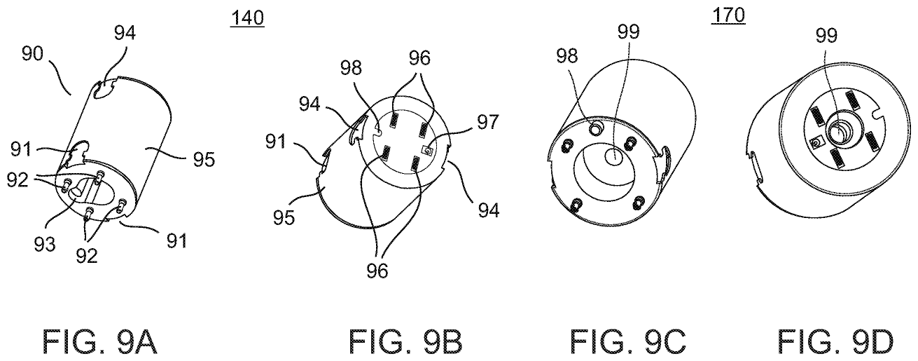

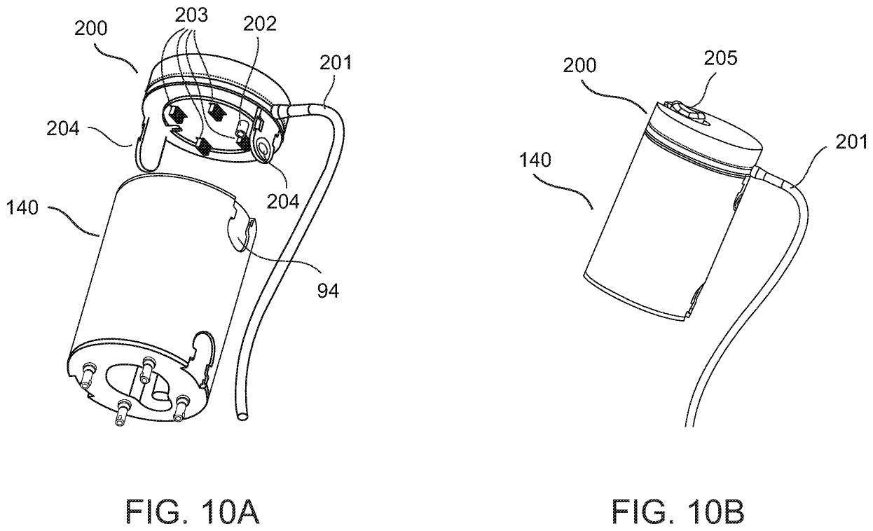

External introducer 12, internal introducer 14 and surgical instrument 16 can each include a motor pack (31, 33, 35 respectively) for actuating deflection of steerable portion (in introducers and instrument) and effector end (in instrument). The motor pack is described in greater detail with reference to FIGS. 9A-10B.

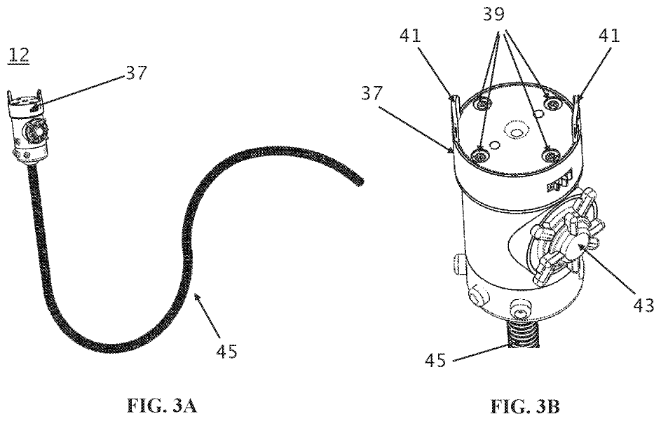

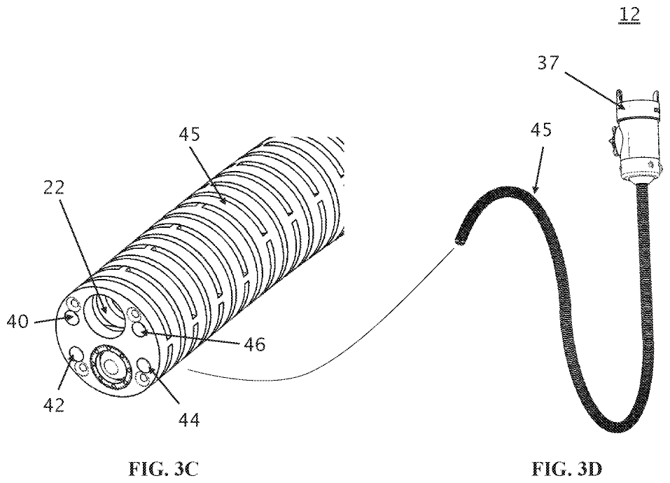

FIGS. 3A-B illustrate the proximal end of external introducer 12 showing motor pack connector 37. Tabs 41 connects mechanically the proximal end of external introducer to the external introducer motor pack (not shown in these Figures). Couplers 39 allows fast connect/release engaging of the motors shafts of an attached motor pack. Connector 37 includes internal gears that are connected to control wires for deflecting the steerable portion, a gear for rotating the introducer with respect to the support frame, and a gear for rotating the leading screw of rail 116 shown in details in FIGS. 5A-B. Motor pack 31, 33 and 35 are each independently controlled by a wired or wirelessly-connected user interface.

External introducer 12 can also include one or more control knobs 43 (two shown) for manually controlling deflection of shaft 45.

As is shown in FIGS. 3C-D, external introducer 12 can include additional lumens 40, 46 which can be used for irrigation, aspiration, and lumens 42, 44 for inserting manually operated of the shelf flexible endoscopic surgical tool or extra small diameter camera or light source. Lumens 40, 42, 44 and 46 have a typical diameter of 2.0-3.2 mm.

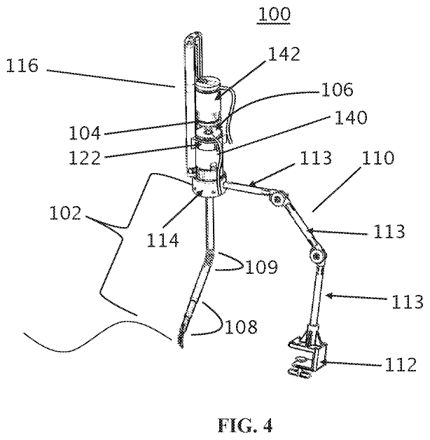

FIG. 4 illustrates another embodiment of the present system which is referred to herein as system 100.

System 100 includes a telescopic introducer 102 and a surgical instrument 104 shown positioned within a central lumen 106 of telescopic introducer 102.

Telescopic introducer 102 includes several sections, a proximal rigid shaft 50-200 mm in length and 5-10 mm in diameter, a steerable portion 20-40 mm in length and 5-10 mm in diameter and a telescopic assembly 50-150 mm in length (when expanded) and 5-10 mm in diameter (tapering distally). Telescopic introducer 102 can be fabricated from an alloy or polymer.

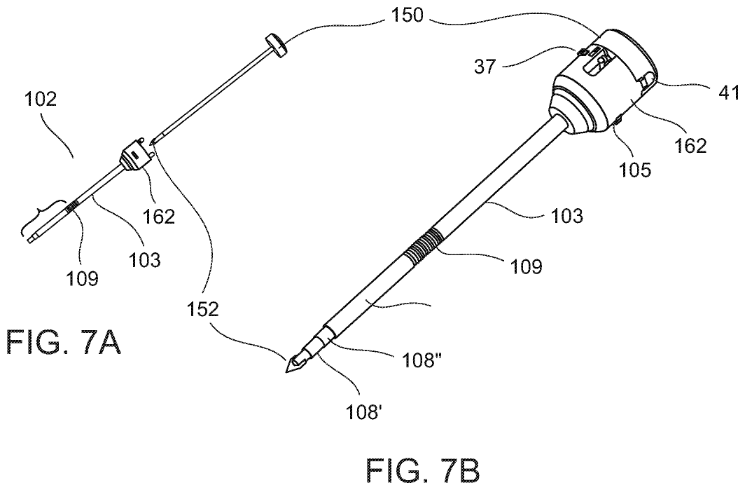

As is shown in FIG. 7A, telescopic introducer 102 includes a shaft 103 having a steerable portion 109 proximal to telescopic portion 108. Telescopic portion 108 includes one or more segments (two shown in FIG. 7B, 108′ and 108″).

System 100 further includes a support frame 110 which is attachable to a fixture (e.g. bed frame) via connector 112. Support frame includes two or more articulating links 113 attached to instrument housing 114.

FIGS. 5A-B illustrate rail 116 in greater detail; FIG. 5A shows rail 116 with cover 117, while FIG. 5B shows the inner mechanism of rail 116.

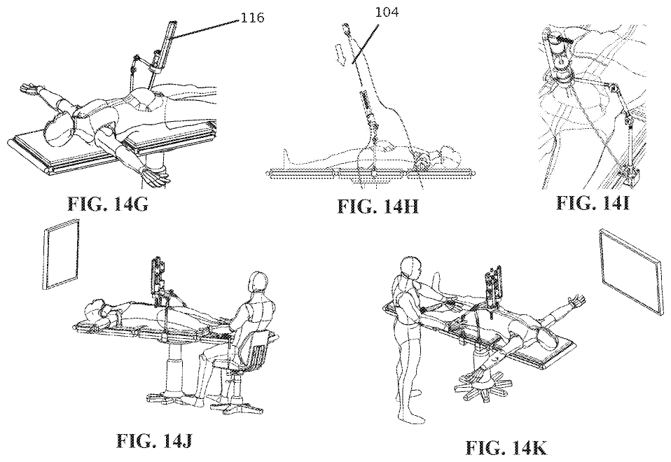

As is mentioned hereinabove, rail 116 provides in/out movement of introducer/instrument with respect to access site. In order to enable such movement, rail 116 includes a rail-mounted bracket 126 that includes a socket 127 which is couplable to a connector (e.g. 205 FIG. 10B) of a motor pack. The connection between a surgical tool and rail bracket 126 is shown in FIG. 14H.

Rail 116 is a mechanical module that moves the entire surgical tool through a linear path. Rail 116, is fixed to the main introducer (e.g. external introducer 12) motor pack via clamp 122. In order to correctly secure rail 116 to introducer housing 160 (shown in FIG. 7A) in the right orientation, snaps 124 are fitted to slots 106 in introducer housing 160, and gear 123 is engaged to gear 37 (FIG. 7B, FIG. 3A) of introducer housing 160.

When gear 37 is rotated by a motor, gear 123 which is fixed to leading screw 125 also rotates. Bracket 126 includes a screw thread fitted to leading screw 125 and 2 linear bearing fitted on smooth rods 128. When leading screw 125 rotates, rods 128 prevents bracket 126 from rotating resulting in linear up/down movement of bracket 126 and corresponding movement from attached instrument/introducer.

Thus, rail 116 enables surgical instrument 104 to move up and down (within telescopic introducer 102). In addition, the shaft of surgical instrument 104 can be deflected via its steerable portion by actuating control wires via the attached motor pack.

Telescopic introducer 102 can be used in laparoscopic procedures through a user-created access site. Such an access site can be created by mounting an access port tool 150 (FIGS. 7A-B) having a cutting distal end 152 within telescopic introducer 102 and using this assembly to puncture through a tissue wall and into a cavity (e.g. through an abdominal wall and into an abdominal cavity). Once the access site is created and telescopic introducer 102 is positioned therethrough, access port tool 150 is removed and telescopic introducer 102 is attached to support frame 110. Motor pack 140 is attached to the introducer, and rail 116 is clamped to the introducer (as shown in FIG. 4). Surgical instrument 104 can then be positioned through central lumen in motor pack 140 and central lumen 154 of telescopic introducer 102, and attached to socket 127 of rail 116. When this setup procedure is completed the system is ready for the surgical procedure. The above described procedure is described in greater detail with reference to FIGS. 14A-H.

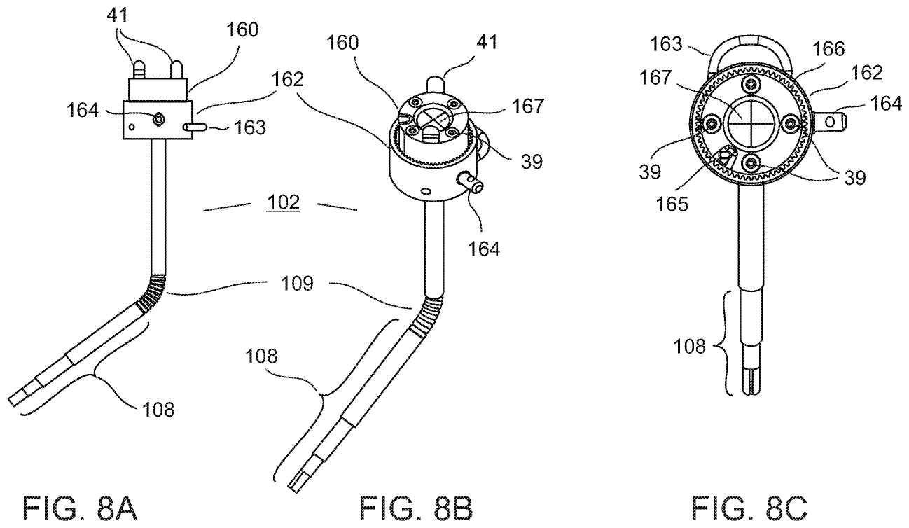

FIGS. 8A-C illustrate telescopic introducer 102 showing telescopic portion 108, steerable portion 109 and motor pack interface 160 in greater detail. Motor pack interface 160 (FIGS. 8A-B) includes connecting tabs 41 that snap into socket 91 in the introducer motor pack (as is shown in FIG. 9A). When clamping motor pack 170 to introducer motor pack interface 160, motor heads 92 (FIG. 9A) engage sockets 39.

Telescopic introducer 102 includes gas valve 165 enabling use thereof in procedures where the cavity is inflated with CO₂. Gas valve 165 includes a seal 167 that allows a shaft of a surgical instrument (e.g. 104) to slide smoothly within the central lumen of introducer 102 while preventing gas leak from the abdominal cavity.

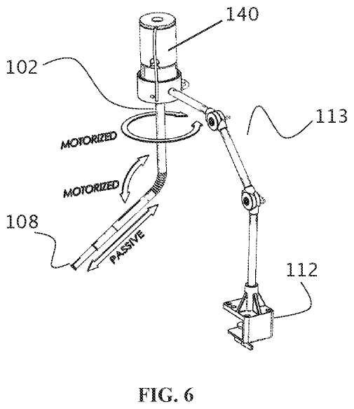

Introducer housing 162 connects the introducer to support frame 110 via plunger 164 and is secured thereto via a U-shaped clamp 163. Clamp 163 allows rotation of introducer housing 162. Gear 166 located at housing 162 is engaged to gear 105 arising from the introducer interface housing 162 (shown in FIG. 7B). When gear 105 is rotated via motor pack 170, introducer 102 rotates with respect to introducer housing 162, resulting in the rotational movement shown in FIG. 6.

Steerable portion 109 enable the articulation shown in FIG. 6. Steering is enabled by cables actuated by a pulley mechanism located in the introducer interface housing 162. The combined movement of rotation of the introducer and deflection of steerable portion 109 allows positioning of an effector end anywhere within a cavity.

FIGS. 9A-D illustrate 2 types of motor packs. Motor pack 140 contains motors that operate surgical instrument 104. Motor heads 92 arises from the lower surface of the motor pack. Keyhole 93 is used to insure that each motor is engaged to its right socket in the surgical instrument. The motor pack includes also (not shown) electronic circuits that enable the control of the motors, communicate with other motor packs or other systems in the operating room, store data, etc. The motor pack also have storage for batteries. The motor pack may function as independent unit that controls the surgical instrument, or may work as part of a system with central control unit. The motor pack may be connected by physical wire to a user interface or may be connected to any number of wireless user interfaces.

Motor pack 140 has a cylindrical shape, with cover 95. Cover 95 includes connecting sockets 94 on upper face and connecting sockets 91 on lower face. As shown in FIG. 9B the upper face of motor pack 140 includes openings for electrical connectors 96 used for communication between the motor pack and the user interface or/and other functions of a robotic system. Power socket 97 supplies power to the motor pack from an external source (e.g. wall connected power supply).

FIGS. 9C-D illustrate a motor pack 170 suitable for use with an introducer (e.g. 12, 14 or 102). This motor pack is similar to the motor pack of FIGS. 9A-B, with the exception that it includes a central lumen 99. Central lumen 99 is continuous with a lumen of an introducer shaft and enables through-insertion of a surgical instrument. Tab 98 is used to ensure correct orientation of the motor pack when connected to the introducer.

FIGS. 10A-B illustrate a motor pack connector module 200. Connector module 200 may be used to supply external power to the motor pack and as communication port between the motor pack and other modules of the robotic system or other systems in the operating room. The connector may be used by the technical support for checking the motor pack and software updates. Connector module 200 serves also as mechanical connector between the surgical instrument to rail 116, by sliding button 205, shown in FIG. 10B. In order to connect the surgical instrument to rail 116, sliding button 205 is clamped into socket 127 in rail 116. FIG. 10A show connecting tabs 204 of connector module 200 that fits into sockets 94 in the upper side of motor pack 140. Electric connectors 203 and external power plug 202 prominent out of the lower surface of connector module 200. External power and data cable 201 supplies external power through plug 202 and data communication through connectors 203.

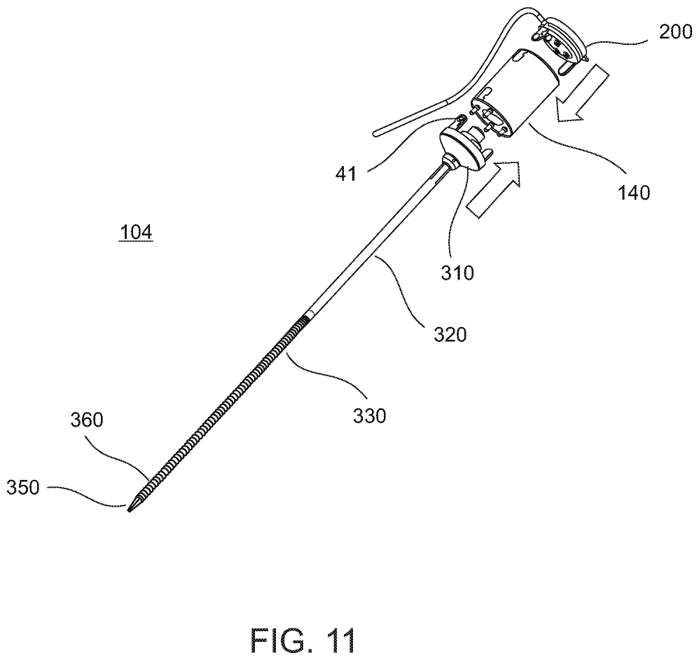

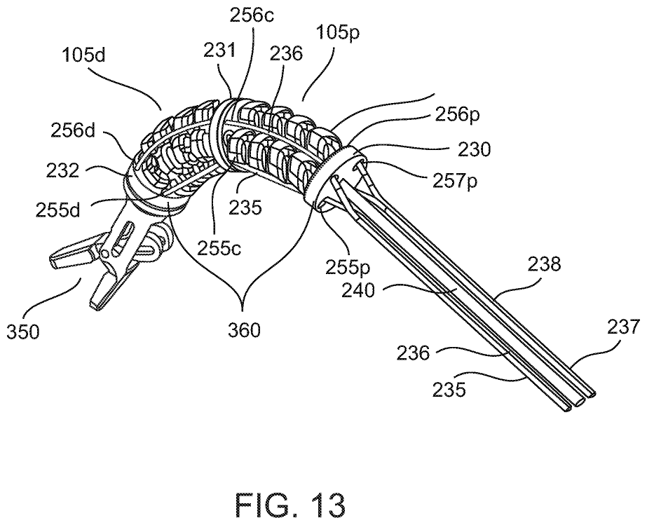

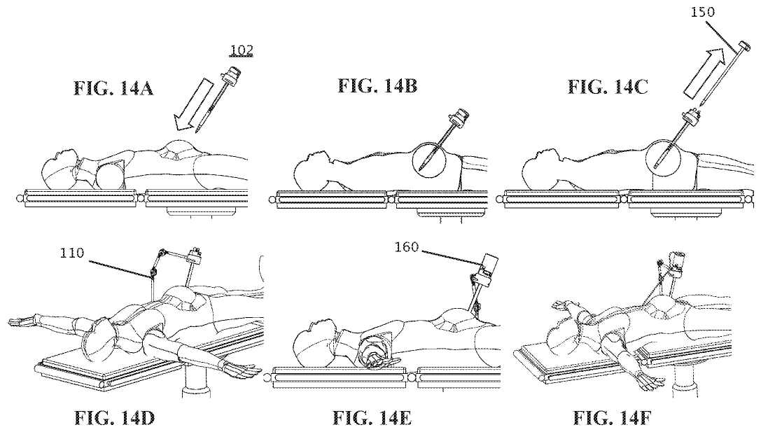

FIG. 11 illustrates surgical instrument 104. The proximal end of the surgical instrument consist of instrument gear housing 310. Rigid shaft 320 arises from the distal end of gear housing 310. Flexible shaft 330 is connected to the distal end of rigid shaft 320. The distal end of the flexible shaft 330 is connected to steerable portion 360. The rigid shaft and the flexible shaft are used to guide the articulation cable from the gear housing to steerable portion 360 as will be described in details in FIG. 13. A cable that actuates end effector 350, runs from gear housing 310 through rigid shaft 320, flexible shaft 330 and steerable portion 360 to the end effector 350 as will be described in details in FIG. 13. Gear housing 310 include mechanisms for pulling articulation cables and for pulling, pushing and rotation of the central cable. Connecting tabs 41 of instrument gear housing 310 engage sockets (94 in FIG. 9A) of motor pack 140.

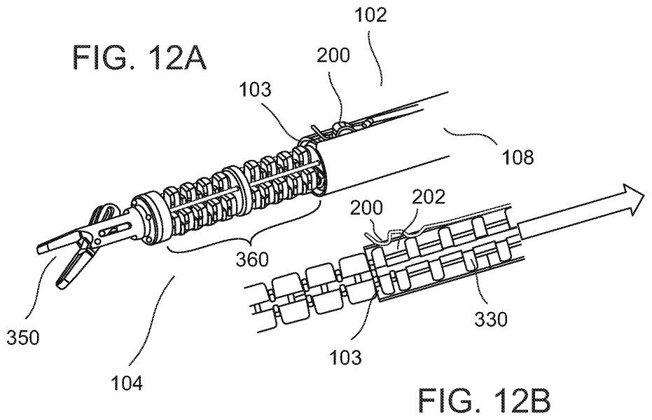

FIGS. 12A-B illustrate telescopic introducer 102 with surgical instrument 104 mounted therein with steerable portion 360 of surgical instrument 104 protruding from opening 103 of telescopic introducer 102. Telescopic portion 108′ can extended and retracted using pull/push wires or via surgical instrument 104 by locking the distal end of shaft 330 of surgical instrument 104 to a distal end of telescopic portion 108′. Such locking can be via a locking mechanism 200 which includes a sprung tab that engages a space between links 202 of shaft 330. When locked, movement of surgical instrument 104 up and down within the lumen of telescopic introducer 102 extends/retracts telescopic portion 108′ and other telescopic tubes 108″-108″″′.

FIG. 13 illustrates steerable portion 360, the cables system that deflect portion 360 and actuates end effector such as needle holder 350, of surgical instrument 104.

Articulation cables 235-238 and central cable 240 are actuated by the mechanism located at the surgical instrument gear housing 310 (FIG. 11). The cables run from gear housing 310 at the proximal end of surgical instrument 104 through rigid shaft 320 and flexible shaft 330 to steerable portion 360 and end effector 350 at the distal end of surgical instrument 104.

Central cable 240 is typically of larger diameter then articulation cables 235-238 since it is used for transferring rotational torque and push/pull forces to end effector 350. Central cable 240 is connected to gear housing 310 and runs through a central lumen at rigid shaft 320, flexible shaft 330 and steerable portion 360. Articulation cables 235-238 are routed radially around the central lumen in rigid shaft 320.

Articulation cables 235-238 are paired in flexible shaft 330. Each pair is located at one side of central lumen of cable 240 as is shown in FIG. 13. The structure of flexible shaft 330 restricts articulation cables 235-238 to follow central cable 240 at a middle portion of the flexible shaft 330. Since the structure of flexible shaft 330 enables bending in one plane only, articulation cables 235-238 do not displace from their routed position when flexible shaft 330 bends, eliminating articulation coupled movement of steerable portion 360. This cables routing approach ensures that when the introducer is bent, steerable portion 360 and end effector 350 do not perform any undesired coupled movement.

Steerable portion 360 may be made of single segment or multiple segments, 2 segments are shown in FIG. 13, proximal segment 360p and distal segment 360d. When cables 235-238 exit the distal end of flexible shaft 330 they are divided through holes 255p-258p in proximal base 230 of steerable portion 360, to upper route (cables 236, 238) and lower route (cables 235, 237). Cables 235-238 come out through holes 255c-258c at central base 231 of steerable portion 360 and connect to distal base 232 of steerable portion 360, through holes 255d-258d.

FIGS. 14A-K illustrate setup and use of system 100 on a patient.

FIG. 14A shows the insertion of an introducer (telescopic introducer 102 shown) into an inflated abdominal cavity. The insertion process is similar to that of a typical trocar: following a small incision made by a surgeon in abdominal wall, introducer 102 is pushed through the cut using access port tool 150 to enlarge the incision to the exact diameter of introducer 102.

Following insertion of introducer 102 to a desired depth (FIG. 14B), the surgeon removes access port tool 150 (FIG. 14C) and connects introducer 102 to a support frame 110 (with bed frame attachment clamp) thereby stabilizing the introducer with respect to the patient body (FIG. 14D). The surgeon then attaches a motor pack 160 to introducer 102 (FIG. 14E), and connects an electrical connector to motor pack 160 (FIG. 14F). A rail 116 is then attached to motor pack 160 (FIG. 14G) and a surgical instrument 104 (such as that shown in FIG. 11) is inserted through motor pack 160 and introducer 102 (FIG. 14H). Surgical instrument 104 is then attached to rail 116 (FIG. 14I).

FIG. 14J illustrates a surgical approach wherein the surgeon controls system 100, a robotic camera and optionally additional robotic instruments while seated. In such a setup the surgeon does not need to be close to the patient bed or even present in the operating room (tele surgery).

FIG. 14K illustrates a surgical approach wherein the surgeon controls system 100, a robotic camera and optionally additional robotic instruments with control interfaces attached to the surgeon's body (e.g. torso/hip). This enables the surgeon to closely monitor the patient during the procedure while being free to move around the operating room, stand by the patient bed and perform additional tasks such as palpating the surgical site, switching surgical instruments or cleaning the camera lens.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

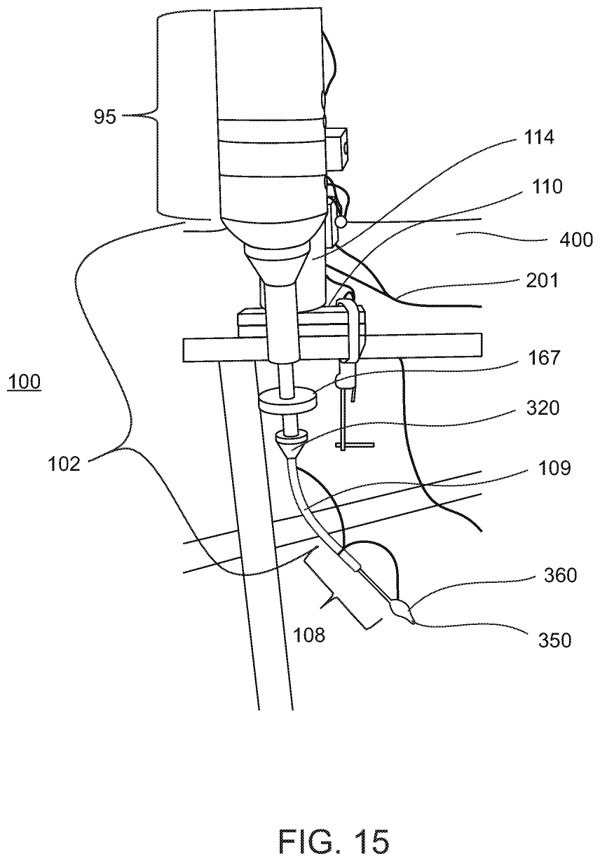

A prototype of a telescopic introducer was developed and manufactured using 3D printing technology (FIG. 15). The prototype includes a cylindrically-shaped motor pack housing (95) printed from an ABS material. The housing has a diameter of 100 mm and a height of 180 mm. The housing includes 2 sets of motors and electrical circuits for controlling the movement of the introducer (102) and a surgical instrument (104) mounted therein. The first motor set actuates the introducer attached at the bottom of the motor housing. The second motor set actuates the internal surgical instrument.

The motors are connected to cables (201) which provide power as well as communication with a user interface. The user interface controls the movement of the introducer and the surgical instrument.

The introducer includes a proximal rigid shaft (320) having a diameter of 13 mm and a length of 150 mm. The steerable portion (109) of the introducer has a diameter of 12 mm and a length of 50 mm. The steerable portion was printed from nylon as a single unitary piece with integrated articulation. The steerable portion has a range of bending of ±110 degrees.

The telescopic assembly (108) includes three tubes each printed from Nylon. Each tube has a wall thickness of 0.8 mm. The external tube of the telescopic portion has an external diameter of 13 mm while the internal tube has an internal diameter of 8 mm. Each of the three tubes is about 60 mm in length allowing a total linear travel of 90 mm.

The surgical instrument is attached to the second motor set which moves the surgical instrument up and down inside the motor pack housing. Since the surgical instrument shaft is attached to the distal tube of the telescopic assembly, such movement extends and retracts the telescopic portion. Additional motors of the second motor set actuates the distal articulation 360 and end effector 350 (a needle holder) of the surgical instrument. A gas valve 167 seals the introducer shaft lumen against the shaft of the surgical instrument.

The surgical instrument includes a rigid stainless steel shaft, having an external diameter of 8 mm and a length of 160 mm. The flexible shaft 330 and distal steerable portion 360 were printed from nylon as a unitary body. The flexible portion 330 has a diameter of 8 mm and a length of 150 mm. The distal steerable portion 360 has a diameter of 7 mm and a length of 25 mm. A tripod (110) secures the prototype introducer-instrument system to a table (400).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for minimally invasive procedures comprising:
(a) a first device having an elongated body including a first lumen having a distal opening, at least a portion of said elongated body being steerable;
(b) a second device being positionable within said first lumen with a distal portion thereof protruding from said distal opening; and
(c) a support frame and a rail being independently couplable to proximal portions of said first device and said second device, wherein said rail is configured such that said second device is movable with respect to said first device along a longitudinal axis of said rail.

2. The system of claim 1, wherein said distal portion of said second device is steerable.

3. The system of claim 2, further comprising a first motor pack attachable to a proximal end of said first device and a second motor attachable to a proximal end of said second device.

4. The system of claim 3, wherein said first motor pack is configured for steering said at least a portion of said elongated body.

5. The system of claim 3, wherein said second motor pack is configured for steering said distal portion of said second device.

6. The system of claim 1, wherein said elongated body is positionable within a body cavity/lumen of a subject through an access site.

7. The system of claim 1, wherein said second device includes a second lumen having a distal opening.

8. The system of claim 7, further comprising a third device having a tool at a distal end thereof, said third device being positionable within said second lumen with said tool protruding from said distal opening of said second lumen.

9. The system of claim 8, wherein said tool is a grasper, needle or a snare.

10. The system of claim 1, wherein said rail includes a linear actuator for moving said second device with respect to said first device along said longitudinal axis.

11. The system of claim 1, wherein said second device includes a tool attached to said distal portion.

12. The system of claim 11, wherein said tool is a grasper, a needle holder or a hook.

13. The system of claim 1, wherein said at least said portion of said elongated body includes at least two independently steerable regions.

14. The system of claim 2, wherein said distal portion is 10-50 mm in length.

15. The system of claim 1, wherein said support frame is attachable to a bed or a floor stand.

16. The system of claim 1, at least one control knob for manually steering said at least said portion of said elongated body.

17. The system of claim 1, wherein said first device includes an irrigation lumen and a suction lumen.

18. An introducer for minimally invasive surgery comprising an elongated body including at least one lumen sized and configured for supporting delivery of a medical device therethrough, wherein a first portion of said elongated body is steerable and a second portion of said elongated body is telescopically extendable and retractable.

19. A system comprising the introducer of claim 18 and the medical device positioned within said at least one lumen.

20. The system of claim 19, wherein the medical device includes a steerable distal portion.

21. The system of claim 20, wherein said distal portion is lockable to said second portion of the introducer, such that when the medical device is moved with respect to the introducer, said second portion of said elongated body is telescopically extended or retracted.
